(19) United States
(12) Patent Application Publication
(10) Pub. No.: US 2008/0306154 A1
(43) Pub. Date: Dec. 11, 2008

(54) Treatment and prevention of major adverse cardiovascular events or major coronary evens by administering Omega-3 fatty acids

(76) Inventors: My Svensson, Vadum (DK); Jeppe Hagstrup Christensen, Aalborg (DK); Erik Berg Schmidt, Skorping (DK); Kaj Anker Jorgensen, Langa (DK)

Correspondence Address: ARENT FOX LLP, 1050 CONNECTICUT AVENUE, N.W., SUITE 400, WASHINGTON, DC, 20036, US

(21) Appl. No.: 11/979,540

(22) Filed: Nov. 5, 2007

Related U.S. Application Data
(60) Provisional application No. 60/856,300, filed on Nov. 3, 2006.

(51) Int. Cl. A61K 31/202; A61P 9/00; A61P 9/10

(52) U.S. Cl. 514/560

(57) ABSTRACT

Omega-3 fatty acid compositions comprising eicosapentaenoic acid (EPA) and docosahexaenoic acid (DHA) are provided, where the compositions are useful for treating, reducing the occurrence of, or preventing major adverse cardiovascular events or major coronary events in patients who have established cardiovascular disease without prior myocardial infarction, preventing their further progression, and treating underlying risk factors for CVD such as hypertension, dyslipidemia, obesity and/or diabetes.

### RELATED APPLICATION DATA

This application claims priority from U.S. Provisional Application No. 60/856,300, which was filed on Nov. 3, 2006, the contents of which are incorporated herein by reference.

### BACKGROUND OF THE INVENTION

1. Field of the Invention

The present invention relates, generally, to compositions comprising omega-3 fatty acids, and particularly to omega-3 fatty acid compositions comprising eicosapentaenoic acid (EPA) and docosahexaenoic acid (DHA), where the compositions are useful for treating and/or preventing major adverse cardiovascular events or major coronary events in patients who have established cardiovascular disease and have not yet suffered from a myocardial infarction (MI). The present invention also includes pharmaceutical formulations made from the compositions, methods of using the formulations to treat or prevent major adverse cardiovascular events or major coronary events in patients who have established cardiovascular disease but have not suffered from an MI, and methods of using the formulations to treat or prevent major adverse cardiovascular events or major coronary events in patients who have established cardiovascular disease but have not suffered from an MI, where the patients are also suffering from any of the various underlying conditions that may lead to major coronary events, including hypertension, dyslipidemia, obesity and/or diabetes.

2. Description of the Related Art

Cardiovascular disease (CVD) is a broad term that encompasses a variety of diseases and conditions. It refers to any disorder in any of the various parts of the cardiovascular system, which consists of the heart and all of the blood vessels found throughout the body. Diseases of the heart may include coronary artery disease, coronary heart disease, cardiomyopathy, valvular heart disease, pericardial disease, congenital heart disease (e.g., coarctation, atrial or ventricular septal defects), and heart failure. Diseases of the blood vessels may include arteriosclerosis, atherosclerosis, hypertension, stroke, aneurysm, peripheral arterial disease, intermittent claudication, vasculitis, venous incompetence, venous thrombosis, varicose veins, and lymphedema. Some patients may have received treatment for their CVD, such as vascular or coronary revascularizations (angioplasty with or without stent placement, or vascular grafting). Some types of cardiovascular disease are congenital, but many are acquired later in life and are attributable to unhealthy habits, such as a sedentary lifestyle and smoking. Some types of CVD can also lead to further heart problems, such as angina, major adverse cardiovascular events and/or major coronary events such as myocardial infarction or coronary intervention, or even death (cardiac or cardiovascular), which underscores the importance of efforts to treat and prevent CVD. See Mayo Clinic Staff, “Cardiovascular disease 101: Know your heart and blood vessels,” available at the website www.mayoclinic.com/health/cardiovascular-disease/HB00032 (2005).

Primary prevention efforts are focused on reducing known risk factors for CVD, or preventing their development, with the aim of delaying, reducing the risk of, or preventing the onset of CVD. Secondary prevention efforts are focused on reducing recurrent CVD and decreasing mortality, major adverse cardiovascular events (MACEs) or major coronary events (MCEs) in patients with established CVD. However, regardless of whether the prevention efforts are characterized as primary or secondary prevention, once a patient has exhibited clinical disease, he or she is at high risk of experiencing MCEs. See Grundy et al., “Primary Prevention of Coronary Heart Disease: Guidance From Framingham,” Circulation 97:1876-1887 (1998).

In the general population, dyslipidemia is a risk factor used to predict the likelihood of developing CVD. In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

Omega-3 fatty acids are known to reduce serum triglycerides by inhibiting diacylglycerol acyltransferase (DGAT) and by stimulating peroxisomal and mitochondrial beta oxidation. Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One form of omega-3 fatty acid is a concentrate of omega-3, long chain, polyunsaturated fatty acids from fish oil containing DHA and EPA that is sold under the trademark Lovaza™ (formerly sold as Omacor®). Such a form of omega-3 fatty acid is described, for example, in U.S. Pat. Nos. 5,502,077, 5,656,667 and 5,698,594, each of which is incorporated herein by reference.

The use of fish oil compositions to prevent cardiovascular events has been investigated in the general population. PCT Published Application No. WO 00/48592 discloses the use of compositions containing omega-3 fatty acids to prevent and/or reduce cardiovascular events in patients who have experienced a prior myocardial infarction.

Various studies have been conducted to determine the effects of dietary fish intake or dietary supplementation with products such as fish oil on risk factors associated with cardiovascular disease. Although some studies have indicated that dietary fish intake or fish oil supplementation may provide beneficial effects, they have not resulted in specific dietary supplementation guidelines or disclosed methods of treating CVD or preventing MCEs.

Some prospective studies suggest that dietary fish intake may lower the chances of death or certain MCEs, such as MI, either in the general population or in women. See, e.g., Nagata et al., Am. J. Epidemiol. 156(9):824-31 (2002); Hu et al., JAMA 287(14):1815-21 (2002); Yuan et al., Am. J. Epidemiol. 154(9):809-16 (2001). Other studies find no evidence for such a conclusion. See, e.g., Morris et al., Am. J. Epidemiol. 142(2):166-75 (1995); Burr et al., Eur. J. Clin. Nutr. 57:193-200 (2003).

Yokoyama et al., Am. Heart J. 146(4):613-620 (2003), examined the effect of daily administration of a combination of 1800 mg of highly purified EPA and an HMG-CoA reductase inhibitor on preventing cardiovascular events in Japanese patients with hyperlipidemia. In the secondary prevention stratum (n=3645), prior MI was present in 28%, stable angina was present in 79%, and other chronic diseases were present in 58% of participants.

Sacks et al., JACC 25(7):1492-98 (1995), provided patients having documented coronary atherosclerosis with 6 g of omega-3 fatty acids daily for 28 months. In comparison to patients in the placebo group, patients in the omega-3 fatty acids group had no significant change in the progression of the coronary atherosclerosis, as measured by change in lumen diameter.

Leng et al., Clin. Nutr. 17(6):265-71 (1998), randomized patients with stable intermittent claudication to receive either a combination of gamma-linolenic acid (280 mg) and eicosapentaenoic acid (45 mg), or a placebo, for two years. Leng et al. observed a nonsignificant (p>0.05) reduction in non-fatal coronary events in the fatty acids group.

Phinney et al., U.S. Patent Publication No. 2005/0137253, discloses that treatment with compositions comprising non-alpha tocopherol and omega-3 fatty acids, such as DHA, are useful for treating inflammatory conditions.

Guidelines for secondary prevention of CVD have been developed based on analysis of evidence from a number of clinical trials, and include smoking cessation, blood pressure control, lipid management, increased physical activity, weight management, diabetes management, antiplatelet/anticoagulant therapy, renin-angiotensin-aldosterone system blocker therapy, and beta-blocker therapy (in patients with a history of prior MI). Those guidelines include a recommendation that all patients should be encouraged to increase consumption of omega-3 fatty acids in the form of fish or capsules (1 g/day), and that patients with elevated triglycerides should receive higher doses for risk reduction, although it was noted that the usefulness of this approach is subject to conflicting evidence and a divergence of opinion. See Smith et al., “AHA/ACC Guidelines for Secondary Prevention for Patients With Coronary and Other Atherosclerotic Vascular Disease: 2006 Update,” J. Am. Coll. Cardiol. 47:2130-2139 (2006).

GISSI-Prevenzione (as described by Marchioli et al. in The Lancet 354:447-455 (1999) and Circulation 105:1897-1903 (2002)) is an 11,324 randomized, controlled study, evaluating the treatment effect of 1 gram/day Omacor (5,666 patients) versus control (5,668 patients). All patients enrolled in this study had a myocardial infaction within the last 3 months and the follow-up period was 3 to 5 years. In this study, Omacor reduced with statistical significance the primary combined efficacy endpoint of death, non-fatal myocardial infarction, and stroke as well as the sudden death secondary endpoint and the cardiovascular death, non-fatal myocardial infarction, and stroke endpoints. Non-fatal cardiovascular events, such as myocardial infarction and stroke were not reduced with statistical significance (140 events in the Omacor group and 144 events in the control group).

Because the development of heart failure is one of the main complications following a myocardial infartion, Macchia et al. (Eur. J. Heart Failure 7:904-909 (2005)) analyzed the effect of Omacor in patients with substantial heart failure (ejection fractions ≦50%). In this sub-population analysis, ejection fraction data to establish left ventricular systolic dysfunction were available for 9630 patients. Compared to patients without substantial heart failure (HF), HF patients (EF≦50%) had higher mortality (12.3% vs. 6.0%) and sudden death (3.4 versus 1.4%) rates. Mortality reduction in HF patients was a significant 24% (p=0.02) versus a non-significant mortality reduction in patients without substantial HF (p=0.17). Sudden death reduction in HF patients was a substantial and significant 58% (p=0.0003) versus an even less-significant sudden death reduction in patients without substantial HF (p=0.71). This sub-analysis established that almost all the benefit in the GISSI-P post-MI population was established in the group with substantial heart failure.

None of the above-mentioned approaches provides compositions and methods for treating and/or preventing major coronary events in patients who have established cardiovascular disease but have not suffered from an MI, or methods of treating underlying risk factors (such as hypertension, dyslipidemia, obesity and/or diabetes) that are associated with development of cardiovascular disease, by administering the compositions of the present invention containing omega-3 fatty acids, and optionally including one or more additional compounds useful in treatment or prevention of cardiovascular disease and/or major adverse cardiovascular events.

### SUMMARY OF THE INVENTION

The present invention meets the unmet needs of the art, as well as others, by providing compositions containing omega-3 fatty acids that preferably include EPA and DHA, preferably Lovaza™ omega-3 fatty acids, that can provide an effective treatment for major coronary events in patients who have established cardiovascular disease without prior myocardial infarction, reduce the occurrence of or prevent their occurrence, as well as treat any of the various underlying conditions that may lead to the development of CVD. These additional underlying conditions may include, but are not limited to, hypertension, dyslipidemia, obesity and/or diabetes. According to a particularly preferred embodiment, the underlying condition is dyslipidemia. According to a further embodiment, the omega-3 fatty acids are provided for co-administration, or as unit doses, with one or more compounds useful for treating CVD, or the underlying conditions responsible for causing CVD, for use in treating, reducing the occurrence of, or preventing major adverse cardiovascular events or major coronary events in patients who have established cardiovascular disease other than a myocardial infarction.

The present invention also provides natural or synthetic omega-3 fatty acids, and/or their pharmaceutically acceptable esters, derivatives, conjugates, precursors or salts, or mixtures thereof, to provide an effective pharmaceutical treatment for major adverse cardiovascular events or major coronary events in patients who have established cardiovascular disease without a prior myocardial infarction, while minimizing unwanted side effects.

One embodiment of the present invention provides a method of utilizing a composition comprising natural or synthetic omega-3 fatty acids, and/or their pharmaceutically acceptable esters, derivatives, conjugates, precursors or salts, or mixtures thereof, in reducing the occurrence of or the prevention of major adverse cardiovascular events or major coronary events in patients who have established cardiovascular disease without a prior myocardial infarction. According to a preferred embodiment, the composition may be utilized to reduce the occurrence of or prevent major coronary events such as myocardial infarctions in this patient population.

Another embodiment of the present invention is a oral formulation of natural or synthetic omega-3 fatty acids or pharmaceutically acceptable esters, derivatives, conjugates, precursors or salts thereof, or mixtures thereof, in the treatment of one or more risk factors for major adverse cardiovascular events or major coronary events in patients who have established cardiovascular disease without a prior myocardial infarction. In one aspect of the embodiment, the risk factors may include hypertension, dyslipidemia, obesity and/or diabetes.

Another embodiment of the invention is a method of treating, reducing the occurrence of and/or preventing major adverse cardiovascular events or major coronary events in patients who have established cardiovascular disease without a prior myocardial infarction, by providing a composition comprising natural or synthetic omega-3 fatty acids or pharmaceutically acceptable esters, derivatives, conjugates, precursors or salts thereof, or mixtures thereof, and thereafter administering the composition to the patient.

Another embodiment of the invention is the use of natural or synthetic omega-3 fatty acids or pharmaceutically acceptable esters, derivatives, conjugates, precursors or salts thereof, or mixtures thereof, for the manufacture of a medicament for treating, reducing the occurrence of or preventing major adverse cardiovascular events or major coronary events in patients who have established cardiovascular disease without a prior myocardial infarction.

The compositions and methods of the present invention may further comprise co-administration of one or more additional compounds useful in the treatment of CVD or one or more of hypertension, dyslipidemia, obesity and/or diabetes. According to a particularly preferred embodiment, the compositions and methods of the present invention are useful in the treatment of dyslipidemia. Also included are unit dosage forms including the omega-3 fatty acids and said one or more additional compounds, and methods for administering same to a patient in need thereof.

In preferred embodiments the pharmaceutical compositions comprise Lovaza™ omega-3 fatty acids, as described in U.S. Pat. Nos. 5,502,077, 5,656,667 and 5,698,594. In other preferred embodiments the pharmaceutical compositions comprise omega-3 fatty acids present in a concentration of at least 40% by weight as compared to the total fatty acid content of the composition. In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:4 to 4:1, more preferably from 1:3 to 3:1, and most preferably from 1:2 to 2:1.

Other novel features and advantages of the present invention will become apparent to those skilled in the art upon examination of the following or upon learning by practice of the invention.

### DETAILED DESCRIPTION OF THE INVENTION

The present invention relates to compositions comprising omega-3 fatty acids, methods of making same, and their use in treating, reducing the occurrence of and/or preventing major adverse cardiovascular events or major coronary events in patients who have established cardiovascular disease without a prior myocardial infarction, as well as their use in treating the underlying risk factors that are associated with CVD. The terms “established cardiovascular disease” or “CVD” include major coronary events (MCEs) which include, but are not limited to, myocardial infarction (MI) and coronary intervention such as coronary revascularization, investigational or interventional angioplasty, percutaneous transluminal coronary angioplasty (PTCA), percutaneous coronary intervention (PCI) and coronary artery bypass graft (CABG); and CVD also includes angina pectoris, documented coronary atherosclerosis, stroke, transient ischemic attack (TIA) and peripheral artery disease (PAD), or any other disorder or condition affecting the heart and/or blood vessels. Risk factors for CVD include hypertension, dyslipidemia, obesity and/or diabetes. Major adverse cardiovascular events (MACE) include cardiac death, other cardiovascular death, MCE, hospitalization for unstable angina, stroke, TIA and hospitalization for PAD. Additional compounds useful in treating, reducing the occurrence of, or preventing CVD or the underlying risk factors associated with CVD may also be beneficially coadministered with the inventive compositions and pharmaceutical formulations, or may be provided in a unit dose form therewith. According to a particularly preferred embodiment, the compositions and methods of the present invention are useful in treating, reducing the occurrence of and/or preventing MCEs such as MI.

### 1. Compositions Containing Omega-3 Fatty Acids

In preferred embodiments, the compositions of the present invention are useful for treating, reducing the occurrence of, or preventing major adverse cardiovascular events or major coronary events in patients who have established cardiovascular disease without a prior myocardial infarction, and the underlying cause(s) thereof. The compositions are preferably useful for reducing the occurrence of or preventing major coronary events such as myocardial infarction (MI) and coronary intervention such as coronary revascularization, investigational or interventional angioplasty, percutaneous transluminal coronary angioplasty (PTCA), percutaneous coronary intervention (PCI) and coronary artery bypass graft (CABG). Such coronary interventions may or may not involve the placement of a bare or drug eluting stent. These inventive compositions preferably comprise Lovaza™ omega-3 fatty acids, as described in U.S. Pat. Nos. 5,502,077, 5,656,667 and 5,698,594. In other preferred embodiments the compositions comprise omega-3 fatty acids present in a concentration of at least 40% by weight as compared to the total fatty acid content of the composition. In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:4 to 4:1, more preferably from 1:3 to 3:1, and most preferably from 1:2 to 2:1. The omega-3 fatty acids may comprise pure EPA or pure DHA.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (EPAX a.s., 1327 Lysaker, Norway).

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The omega-3 fatty acids can be present in an amount from about 500 mg to about 10 grams, more preferably about 1000 mg to about 6 grams, and most preferably from about 1500 mg to about 4 grams. A particularly preferred amount of omega-3 fatty acids is about 2000 mg (about 2 grams). This amount may be provided in one or more dosage forms, preferably one dosage form. The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is Lovaza™ (omega-3 acid ethyl esters) (K85EE, Pronova BioPharma A.S., Lysaker, Norway) and preferably comprises the following characteristics (per dosage form):

| Test | Minimum Value | | Maximum Value |
|---|---|---|---|
| Eicosapentaenoic acid C20:5 EE | 430 | mg/g | 495 mg/g |
| Docosahexaenoic acid C22:6 EE | 347 | mg/g | 403 mg/g |
| EPA-EE and DHA-EE | 800 | mg/g | 880 mg/g |
| Total n-3 fatty acids | 90% | (w/w) | |

The active ingredient of the present invention, omega-3 fatty acids, may be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

The omega-3 fatty acids may optionally be co-administered with one or more additional compounds, or provided in a unit dose pharmaceutical formulation with one or more additional compounds, where those additional compounds are useful in reducing the occurrence of or preventing CVD from occurring or progressing, are useful in reducing the occurrence of or preventing major adverse cardiovascular events or MCEs, or are effective in treating any of the underlying risk factors that are commonly associated with CVD.

The additional compounds in accordance with the present invention may be selected from the group consisting of angiotensin-converting-enzyme (ACE) inhibitors; angiotensin receptor blockers (ARBs); renin inhibitors, dyslipidemic agents such as HMG CoA reductase inhibitors (statins) (preferably including, but not limited to, pitavastatin, simvastatin, rosuvostatin, pravastatin, atorvastatin, lovastatin, and fluvastatin); dihydropyridine calcium channel blockers (preferably including, but not limited to, Bay K 8644, amlodipine, felodipine, lacidipine, lercanidipine, nicardipine, nifedipine, nimodipine, nisoldipine, nitrendipine and isradipine; antiarrhythmic agents (preferably including, but not limited to, quinidine, procainamide, disopyramide, lidocaine, mexiletine, tocainide, phenytoin, encainide, flecainide, moricizine, propafenone, esmolol, propranolol, acebutolol, metoprolol, amiodarone, azimilide, bretylium, clofilium, dofetilide, ibutilide, sematilide, sotalol, verapamil, mebefradfil, diltiazem adenosine, and digoxin); azetidinone-based cholesterol absorption inhibitors (preferably including, but not limited to, ezetimibe, MD-0727, and SCH60663); niacin and derivatives (preferably including, but not limited to, nicotinamide); PPAR agonists/antagonists (preferably including, but not limited to, PPAR-alpha, PPAR-gamma, PPAR-delta, PPAR-alpha/gamma, PPAR-gamma/delta, PPAR-alpha/delta, and PPAR-alpha/gamma/delta agonists and antagonists, as well as partial agonists and/or antagonists, including but not limited to fibrates such as fenofibrate, tesaglitazar, naviglitizar, mivaglitazar and thiazolidinediones such as pioglitazone and rosiglitazone); bile acid sequestrants (preferably including, but not limited to, cholestyramine, cholestipol, and colesevelam); antiplatelet drugs (including, but not limited to, aspirin, clopidogrel, and ticlopidine); and pharmaceutically acceptable esters, derivatives, conjugates, precursors or salts thereof, and mixtures thereof.

Also envisioned in accordance with the present invention is the use of similar compounds to those set forth above, which may be discovered in the future, or already existing compounds that may be approved for new uses in the future. Further envisioned is the optional inclusion of additional compounds useful in the treatment of conditions such as hypertension and diabetes, which are known to contribute to cardiovascular disorders.

Where provided, these optional additional active ingredients are useful in the treatment of major adverse cardiovascular events or major coronary events in patients who have established cardiovascular disease without a prior myocardial infarction, or any of the underlying risk factors or diseases that cause CVD. These may include, but are not limited to, hypertension, dyslipidemia, obesity and/or diabetes.

The optional additional active ingredients, when provided, are including in amounts that are sufficient to provide treat, reduce the occurrence of, and/or prevent CVD, reduce the occurrence of or prevent major adverse cardiovascular events or major coronary events in patients who have established cardiovascular disease without a prior myocardial infarction, and/or treat the underlying risk factors that are elevating the risk of CVD. The optional additional ingredients are provided in amounts that are generally regarded as safe, and are effective in treating, reducing the occurrence of and/or preventing CVD and treating its underlying causes.

The composition comprising concentrated omega-3 fatty acids may be prepared in the form of a capsule, such as a hard gelatin capsule; a tablet; a powder that can be dispersed in a beverage; a liquid; or a soft gel capsule. The composition may also be contained in a liquid suitable for injection or infusion. However, the methods of preparing the inventive compositions for administration are not to be limited to any particular dosage form. Rather, they may be prepared as any pharmaceutically acceptable dosage form, including other solid oral dosage forms, other liquid oral dosage forms, and any other suitable dosage forms. When provided, the one or more optional additional active ingredients may also be provided in the dosage form, as a homogeneous solution or a heterogenous suspension with a solvent comprising the omega-3 fatty acids, so as to create a convenient unit dose form, or a soft gelatin capsule coated with the one or more optional additional active ingredients (see U.S. Provisional Patent Application No. 60/780,306, hereby incorporated by reference).

In some embodiments, the unit dose formulations of the present invention allow for improved effectiveness of each active ingredient, with one or both administered as a conventional full-strength dose, as compared to the formulations in the prior art. In other embodiments, the formulations of the present invention may allow for reduced dosages of the optional additional ingredients, such as 25-80% of the conventional full-strength dose, as compared to the formulations in the prior art, while still maintaining or even improving upon the effectiveness of each active ingredient.

The present combinations of concentrated omega-3 fatty acids and one or more additional ingredients, taken from the list set forth above, may allow for a greater effect than any expected combined or additive effect of the compounds alone. Thus, the combined treatment using the active ingredients, separately or through the novel combination product of the present invention, may cause an unexpected increase in effect of the active ingredients. This may allow increased effectiveness with standard dosages, or, alternatively, may allow maintained effectiveness with reduced dosages of the active ingredients.

2. Methods of Treating, Reducing the Occurrence of and/or Preventing Major Coronary Events in Patients who Have Established Cardiovascular Disease Without a Prior Myocardial Infarction

The compositions containing omega-3 fatty acids described above can be administered in a daily amount of from about 0.1 g to about 10 g, more preferably about 1 g to about 6 g, and most preferably from about 1.5 g to about 4 g, to patients who have established CVD other than a myocardial infarction, in order to treat, reduce the occurrence of and/or prevent major adverse cardiovascular events or MCEs from developing or progressing. According to a particularly preferred embodiment of the present invention, about 2 g of omega-3 fatty acids are administered daily.

The daily dosages of concentrated omega-3 fatty acids can be administered together in from 1 to 10 dosages, with the preferred number of dosages from 1 to 4 times a day, most preferred 1 to 2 times a day. The administration is preferably oral administration, although other forms of administration that provide a unit dosage of concentrated omega-3 fatty acids may be used. The administration of the dosages is preferably effective in treating, reducing the occurrence of and/or preventing major adverse cardiovascular events or major coronary events in patients who have established cardiovascular disease without a prior myocardial infarction, and/or is effective in treating the underlying risk factor(s) responsible for causing the CVD in the patient, particularly hypertension, dyslipidemia, obesity and/or diabetes.

The compositions containing omega-3 fatty acids can optionally be co-administered with one or more additional compounds useful for alleviating CVD in patients who have established CVD without a prior myocardial infarction, and/or is effective in treating the underlying risk factor(s) responsible for causing the CVD in such a patient, particularly hypertension, dyslipidemia, obesity and/or diabetes. Administration of unit dose forms of the omega-3 fatty acids and one or more additional compounds, as listed above, is also contemplated in accordance with the present invention.

In one embodiment, treatment in accordance with the present invention normalizes blood lipid levels and/or reduces systolic blood pressure levels to under 140 mmHg and/or reduces diastolic blood pressure levels to under 90 mmHg. Blood lipid levels may be measured in accordance with any accepted method in the art. Blood pressure levels may be measured in accordance with any recognized method in the art, including, but not limited to, supine, sitting, upright, standing or 24-hr ambulatory.

### EXAMPLE

### Secondary Prevention Study in 206 CVD patients

In a study conducted by Svensson et al., Clin. J. Am. Soc. Nephrol. 1:780-786 (2006), patients with established CVD who had undergone at least six months of stable hemodialysis treatment were assessed for inclusion in a study to determine the effect of fish oil as secondary prevention for CVD. Of 717 patients assessed for eligibility, 206 were selected and randomized into a treatment group (n=103) that received 2 capsules of Omacor® (total dose of 1.7 g/day omega-3 fatty acids) daily, and a placebo group (n=103) that received 2 capsules containing olive oil. Of the 206 patients in this study, 139 patients had not had a prior acute myocardial infarction (AMI) at the time of enrollment. Of these 139 non-AMI patients, 63 were randomized to Omacor while 76 ended up in the placebo treatment group. Due to the small number of events, the statistical analysis described below uses the Chi-square Test, or Fisher Exact Test if one of the treatment groups has less than 5 events, to establish the statistical significance of the treatment effect. The patients were followed for two years.

TABLE 1
Number of Patients by Type and Treatment

| | Treated with | Treated with |
|---|---|---|
| | Omacor | Placebo |
| All patients | 103 | 103 |
| Patients with Prior MI | 40 | 27 |
| Patients with NO Prior MI | 63 | 76 |

As tables 2 and 3 demonstrate, and as expected based upon the known art from the GISSI-Prevenzione study, in patients with a prior acute MI (AMI), Omacor substantially reduces the rate of MCE and AMI. In this study, the MCE rate reduction by Omacor is 78% with near-statistical significance and the AMI rate reduction by Omacor is 87% with statistical significance.

TABLE 2
Study Results for MCE endpoint - Patients with Prior AMI

| | number of | number of | | Relative Rate |
|---|---|---|---|---|
| | patients | patients with | Event rate | Reduction |
| | treated | MCE event | (%) | (%) |
| Omacor | 40 | 2 | 5.0% | 77.5% |
| Placebo | 27 | 6 | 22.2% | XXXXXXXX |

Fisher Exact Test p-value 0.053
Likelyhood Ratio Chi-square 4.523

TABLE 3
Study Results for AMI endpoint - Patients with Prior AMI

| | number of | number of | | Relative Rate |
|---|---|---|---|---|
| | patients | patients with | Event rate | Reduction |
| | treated | MCE event | (%) | (%) |
| Omacor | 40 | 1 | 2.5% | 86.5% |
| Placebo | 27 | 5 | 18.5% | XXXXXXXX |

Fisher Exact Test p-value 0.0353
Likelyhood Ratio Chi-square 5.1736

Surprisingly, in the patient group without a previous AMI (tables 4 and 5), Omacor also reduces the cardiovascular event rate. Although not reaching statistical significance, treatment with Omacor achieves a substantial reduction of the MCE rate (45%) and the AMI rate (55%). This is unexpected because AMI is a typical cause of the development of heart failure due to the partial necrosis of heart failure upon reperfusion injury near the site of the coronary infarction. The overall benefit established with 1 gram of Omacor in GISSI-Prevenzione was almost entirely established in patients suffering from a substantial degree of heart failure in the sub-population with a left ventricular ejection franction of <50% (see Macchia reference).

TABLE 4
Study Results for MCE endpoint - Patients with NO Prior AMI

| | number of | number of | | Relative Rate |
|---|---|---|---|---|
| | patients | patients with | Event rate | Reduction |
| | treated | MCE event | (%) | (%) |
| Omacor | 63 | 5 | 7.9% | 45.2% |
| Placebo | 76 | 11 | 14.5% | XXXXXXXX |

Chi-square Test p-value NS
Likelyhood Ratio Chi-square 1.4869

TABLE 5
Study Results for AMI endpoint - Patients with NO Prior AMI

| | number of | number of | | Relative Rate |
|---|---|---|---|---|
| | patients | patients with | Event rate | Reduction |
| | treated | MCE event | (%) | (%) |
| Omacor | 63 | 3 | 4.8% | 54.8% |
| Placebo | 76 | 8 | 10.5% | XXXXXXXX |

Fisher Exact Test p-value NS
Likelyhood Ratio Chi-square 1.641

From this study, it is clear that a much higher dose of 2 grams of Omacor per day did provide a benefit in patients without a prior MI and associated typical low rates of heart failure. In the GISSI-Prevenzione study, patients a without substantial degree of heart failure (left ventricular ejection fractions more than 50%) did not receive a significant benefit from 1 gram per day Omacor treatment (see Macchia reference).

Although the therapeutic benefit of Omacor in patients without a prior AMI is not as large as the benefit in patients with a prior AMI, the relative values for the Likelihood Ratio Chi-square values demonstrate a clear contribution of the patients without prior AMI to the overall statistical significance of the study results. Tables 2 and 4 indicate a relative contribution of the non-MI patients to the statistical significance of the MCE benefit in the overall population of almost 25% (MCE: 100%×[1.4869/(1.4869+4.523)]; AMI:100%×[1.64/(1.641+5.1736)]).

Despite not achieving statistical significance in patients with no prior AMI, tables 6 and 7 clearly demonstrate how the “no prior AMI patients” contribute substantially to the statistical significance of the Omacor treatment effect in the overall patient population versus the patients in the “prior AMI” sub-population. The smaller p-values in tables 6 and 7 demonstrate a more robust statistical significance versus the p-values in tables 2 and 3 respectively.

TABLE 6
Study Results for MCE endpoint - All patients

| | number of | number of | | Relative Rate |
|---|---|---|---|---|
| | patients | patients with | Event rate | Reduction |
| | treated | MCE event | (%) | (%) |
| Omacor | 103 | 7 | 6.8% | 58.8% |
| Placebo | 103 | 17 | 16.5% | XXXXXXXX |

Chi-square Test p-value 0.0299

TABLE 7
Study Results for AMI endpoint - All patients

| | number of | number of | | Relative Rate |
|---|---|---|---|---|
| | patients | patients with | Event rate | Reduction |
| | treated | MCE event | (%) | (%) |
| Omacor | 103 | 4 | 3.9% | 69.2% |
| Placebo | 103 | 13 | 12.6% | XXXXXXXX |

Chi-square Test p-value 0.0227

In fact, in addition to the unexpected Omacor benefit found in patients without a prior MI, this study also demonstrates a positive effect of 2 grams per day Omacor treatment on the rate of MCE and MI in post-MI patients. Despite its large size, at 1 gram of Omacor per day the GISSI-Prevenzione study demonstrated only a marginal impact on the MCE and MI event rate.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

What is claimed:

1. A method of reducing the occurrence of or preventing a major adverse cardiovascular event (MACE) or major coronary event (MCE) in a patient who has established cardiovascular disease without prior myocardial infarction, comprising the steps of:
providing an omega-3 fatty acid composition comprising eicosapentaenoic acid (EPA) and docosahexaenoic acid (DHA); and
administering the composition to the patient in an amount effective to reduce the occurrence of or prevent a MACE or an MCE.

2. A method of treating CVD in a patient who has established cardiovascular disease without prior myocardial infarction, comprising the steps of
providing an omega-3 fatty acid composition comprising eicosapentaenoic acid (EPA) and docosahexaenoic acid (DHA); and
administering the composition to the patient in an amount effective to treat CVD.

3. The method of claim 2, wherein the method provides an effective treatment for underlying conditions or risk factors of major adverse cardiovascular events or major coronary events.

4. The method of claim 2, wherein the method reduces the occurrence of or prevents the occurrence of a major adverse cardiovascular event or major coronary event in a patient who has established cardiovascular disease without prior myocardial infarction.

5. The method of claim 4, wherein the major coronary event is selected from the group consisting of myocardial infarctions; and coronary interventions selected from the group consisting of coronary revascularization, investigational or interventional angioplasty, percutaneous transluminal coronary angioplasty, percutaneous coronary intervention, and coronary artery bypass graft.

6. The method of claim 2, wherein the method reduces the occurrence of or prevents myocardial infarction in a patient who has established cardiovascular disease without prior myocardial infarction.

7. The method of claim 2, wherein the method provides an effective treatment for conditions selected from the group consisting of hypertension, dyslipidemia, obesity, and diabetes.

8. The method of claim 2, wherein the omega-3 fatty acid composition comprises omega-3 fatty acids present in a concentration of at least 40% by weight as compared to the total fatty acid content of the composition.

9. The method of claim 2, wherein the omega-3 fatty acid composition comprises omega-3 fatty acids present in a concentration of at least 80% by weight as compared to the total fatty acid content of the composition.

10. The method of claim 2, wherein the omega-3 fatty acid composition comprises at least 50% by weight of EPA and DHA as compared to the total fatty acid content of the composition.

11. The method of claim 2, wherein the omega-3 fatty acid composition comprises at least 80% by weight of EPA and DHA as compared to the total fatty acid content of the composition.

12. The method of claim 2, wherein the omega-3 fatty acid composition comprises about 5% to about 95% by weight of EPA as compared to the total fatty acid content of the composition.

13. The method of claim 2, wherein the omega-3 fatty acid composition comprises about 40% to about 55% by weight of EPA as compared to the total fatty acid content of the composition.

14. The method of claim 2, wherein the omega-3 fatty acid composition comprises about 5% to about 95% by weight of DHA as compared to the total fatty acid content of the composition.

15. The method of claim 2, wherein the omega-3 fatty acid composition comprises about 30% to about 60% by weight of DHA as compared to the total fatty acid content of the composition.

16. The method of claim 2, wherein the omega-3 fatty acid composition is provided as a daily dose of from about 1.5 to about 4 grams per day.

17. The method of claim 20, wherein the omega-3 fatty acid composition is provided in a daily dose of about 2 grams per day.

18. The method of claim 2, wherein the omega-3 fatty acid composition comprises omega-3 fatty acids selected from the group consisting of omega-3 polyunsaturated, long-chain fatty acids; esters of omega-3 fatty acids with glycerol; esters of omega-3 fatty acids and a primary, secondary or tertiary alcohol; and mixtures thereof.

19. The method of claim 2, wherein the EPA and DHA are in a weight ratio of EPA:DHA of from 4:1 to 1:4.

20. The method of claim 2, wherein the EPA and DHA are in a weight ratio of EPA:DHA of from 1:2 to 2:1.

21. The method of claim 2, wherein the omega-3 fatty acid composition is provided for co-administration with one or more additional compounds selected from the group consisting of angiotensin-converting-enzyme inhibitors; angiotensin receptor blockers; renin inhibitors; HMG CoA reductase inhibitors; dihydropyridine calcium channel blockers; antiarrhythmic agents; azetidinone-based cholesterol absorption inhibitors; niacin and derivatives; PPAR agonists/antagonists; bile acid sequestrants; antiplatelet drugs; and pharmaceutically acceptable esters, derivatives, conjugates, precursors or salts thereof.

22. The method of claim 21, wherein the omega-3 fatty acid composition is administered apart from administration of the one or more additional compounds.

23. The method of claim 21, wherein the omega-3 fatty acid composition is administered simultaneous to the administration of the one or more additional compounds.

24. The method of claim 21, wherein the omega-3 fatty acid composition and the one or more additional compounds are administered in a concomitant treatment regime.

25. The method of claim 21, wherein the omega-3 fatty acid composition and the one or more additional compounds are administered together in a unit dose form.
